(19) United States
(12) Patent Application Publication
(10) Pub. No.: US 2020/0297545 A1
(43) Pub. Date: Sep. 24, 2020

(54) DEVICE FOR FORMING A COMPOSITE WEB OF AN ABSORBENT PAD FOR ABSORBENT ARTICLES

(71) Applicant: GDM S.p.A., Bologna (IT)

(72) Inventors: Marco ROSANI, Vailarate (Cremona) (IT); Matteo PIANTONI, Albino (Bergamo) (IT); Francesco GIARDINI, Romano di Lombardia (Bergamo) (IT)

(21) Appl. No.: 16/822,606

(22) Filed: Mar. 18, 2020

(30) Foreign Application Priority Data
Mar. 21, 2019 (IT) 102019000004097

(51) Int. Cl. A61F 13/15; A61F 13/51; B29C 48/00; B32B 5/02

(52) U.S. Cl. CPC A61F 13/15699; A61F 13/15764; A61F 13/51; B29C 48/0018; B29L 2031/4878; A61F 2013/15926; A61F 2013/51026; A61F 2013/51038; B32B 5/022

(57) ABSTRACT

This invention relates to a device for forming a composite web of an absorbent pad for absorbent articles, such as diapers, comprising an extruding means for extruding a thermoplastic material and a delivery means for delivering superabsorbent polymer material; a suction forming surface is configured to receive the filaments of thermoplastic material blown out of the one or more delivery mouths of the extruding means and to receive the superabsorbent polymer material coming out of the delivery means, to make a composite, meltblown web comprising the superabsorbent polymer material; the delivery means has at least one outlet mouth for feeding superabsorbent polymer material and configured to be movable at least from a first distance to a second distance, or vice versa, relative to the delivery mouth of the extruding means in such a way as to vary the concentration of the superabsorbent polymer material in the composite web.

This application claims priority to Italian Patent Application 102019000004097 filed Mar. 21, 2019, the entirety of which is incorporated by reference herein.

### BACKGROUND OF THE INVENTION

This invention relates to a device for forming a composite web of an absorbent pad for absorbent articles.

The composite web of this device is a web comprising “meltblown” fibers, that is to say, a composite web having at least one layer obtained by melt blowing.

In this field of application, the need was felt to control the concentration of superabsorbent polymer material particles fed into the layer of meltblown fibers.

### SUMMARY OF THE INVENTION

In this context, one aspect of this invention is to provide a device for forming a composite web of an absorbent pad for absorbent articles, such as diapers, for example, and which comprises an extruding means for extruding a thermoplastic material and provided with one or more channels, each having a respective delivery mouth for delivering filaments of thermoplastic material.

A delivery means for delivering superabsorbent polymer material has at least one outlet mouth for feeding the superabsorbent polymer material into the flow of filaments of thermoplastic material coming out of the extruding means.

A suction forming surface is configured to receive the filaments of thermoplastic material coming out of the extruding means and comprising the superabsorbent polymer material, to make a composite, meltblown web comprising meltblown thermoplastic fibers and superabsorbent polymer material.

The outlet mouth of the delivery means is configured to be movable at least from a first distance to a second distance, or vice versa, relative to the delivery mouth of the extruding means in such a way as to vary the concentration of the superabsorbent polymer material in the composite web.

### BRIEF DESCRIPTION OF THE DRAWINGS

Further features of the invention and its advantages are more apparent in the following non-limiting description of preferred but non-exclusive embodiments of a device for forming a composite web of an absorbent pad for absorbent articles, as illustrated in the accompanying drawings, in which:

FIG. 1 is a schematic front view of a first embodiment of the device according to this invention;

FIG. 2 is a schematic front view of a second embodiment of the device according to this invention.

### DESCRIPTION OF THE PREFERRED EMBODIMENTS

The reference numeral 1 denotes a device for forming a composite web of an absorbent pad for absorbent articles according to this invention.

The term “absorbent articles” is used to mean diapers for children and/or adults.

The device 1 comprises a tank 2 for containing thermoplastic material.

By way of non-limiting example, the thermoplastic material may be polypropylene, polyethylene, acrylic resin, polyamide (nylon, for example), polystyrene, polyvinyl chloride (PVC), acrylonitrile butadiene styrene (ABS), polycarbonate.

A substantially hydrophilic polymeric material, such as hydroxyl-polypropylene for example, may also be used.

An extruding means 3 for extruding thermoplastic material fed from the tank 2 comprises one or more channels, each having a respective delivery mouth 4 for delivering filaments 5 of thermoplastic material.

The extruding means 3 is configured to produce a flow of filaments 5 of meltblown thermoplastic material.

A source 6 of heated, pressurized gaseous fluid is in fluid communication with the extrusion ducts of the extruding means 3.

A delivery means 9 for delivering superabsorbent polymer material 10 is configured to feed the superabsorbent polymer material 10 into the flow of filaments 5 of thermoplastic material coming out of the extruding means 3.

In a variant embodiment, a pressurized fluid source 20 is connected to the delivery means 9 so that the superabsorbent polymer material 10 is fed under pressure into the flow of filaments 5 of thermoplastic material coming out of the extruding means 3.

A suction forming surface 7 is configured to receive the flow of filaments 5 of thermoplastic material coming out of the extruding means 3 and to receive the superabsorbent polymer material 10 coming out of the delivery means 9, to make a composite web 8 of meltblown fibers comprising the superabsorbent polymer material 10, or composite meltblown web 8.

In this embodiment, the suction forming surface 7 is the peripheral surface of a suction roller 12 that rotates about its axis 12a.

The delivery means 9 is provided with at least one outlet mouth 11 for feeding the superabsorbent polymer material 10.

Optionally, the delivery means 9 for delivering the superabsorbent polymer material 10 may be provided with a means for heating the superabsorbent polymer material 10.

The outlet mouth of the delivery means is configured to be movable at least from a first distance “d1” to a second distance “d2”, or vice versa, relative to the delivery mouth 4 of the extruding means 3 in such a way as to vary the concentration of the superabsorbent polymer material 10 in the meltblown composite web 8.

This distance variation allows the outlet mouth 11 of the delivery means 9 to be positioned near or far from the delivery mouth 4 of the extruding means 3 so as to vary the concentration of the superabsorbent polymer material 10 in the meltblown composite web 8.

More specifically, by positioning the outlet mouth 11 of the delivery means 9 near the delivery mouth 4 of the extruding means 3 at a distance d1, the concentration of the superabsorbent polymer material 10 in the flow of filaments 5 of thermoplastic material is greater than when the outlet mouth 11 of the delivery means 9 is positioned far from the delivery mouth 4 of the extruding means 3, at a distance d2 greater than d1.

That is because the temperature of the flow of filaments 5 of thermoplastic material decreases from the delivery mouth 4 of the extruding means 3 to the forming surface 7 of the composite web 8; hence, the superabsorbent polymer material 10 adheres to the flow of filaments 5 of thermoplastic material more when it is closer to the delivery mouth 4 of the extruding means 3 than it is to the forming surface 7 of the meltblown composite web 8.

The device 1 comprises a first feed unit 13 for feeding a first continuous web 14 of non-woven fabric, preferably spunbond, disposed upstream of the forming surface 7, in the feed direction of the meltblown composite web 8, so that the meltblown composite web 8 is made on the first continuous web 14, as illustrated in FIG. 2, held by suction on the forming surface 7.

The presence of the first feed unit 13 for feeding the first continuous web 14 of non-woven fabric is optional.

In the second embodiment, shown in FIG. 2, the device comprises a second feed unit 15 for feeding a second continuous web 16 of non-woven fabric, preferably spunbond, disposed downstream of the forming surface 7, in the feed direction of the meltblown composite web 8, so that the second continuous web 16 is disposed on the meltblown composite web 8 made on the forming surface 7.

The presence of the second feed unit 15 for feeding the second continuous web 16 of non-woven fabric is optional.

If the device 1 comprises both the first feed unit 13 and the second feed unit 15, the meltblown composite web 8 is interposed between the first continuous web 14 and the second continuous web 16 to define a continuous multilayer web 19.

In a variant embodiment, the device 1 comprises a feeding means 17 for feeding a moistening substance 18 and disposed downstream of the second feed unit 15, in the feed direction of the second continuous web 16.

That way, the moistening substance 18 is delivered to the second continuous web 16.

Alternatively, in a variant not illustrated, a moistening substance 18 is fed to the extruding means 3 so that the flow of filaments comprises the moistening substance when it comes out of the extruding means 3.

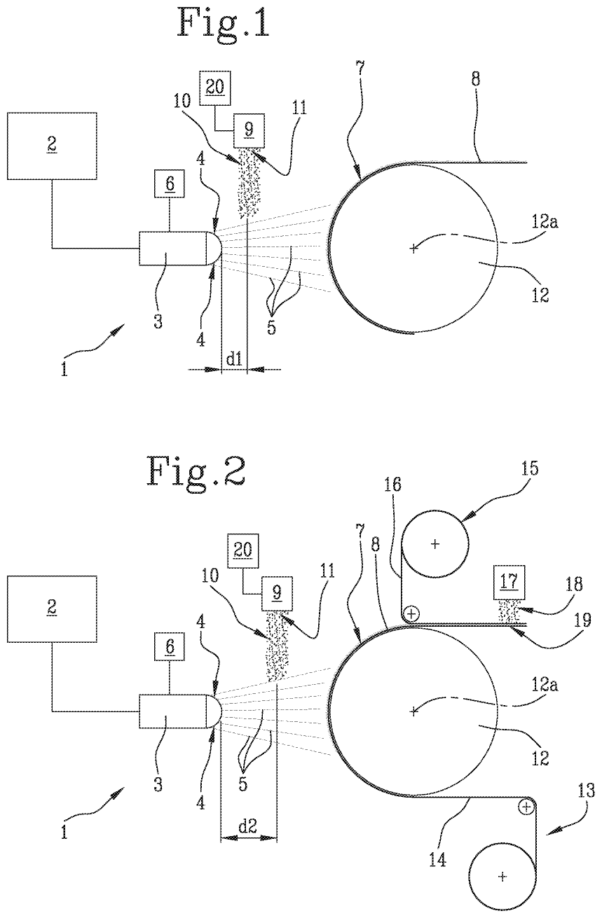

In the first embodiment of the invention, shown in FIG. 1, only the meltblown composite web 8 comprising superabsorbent polymer material 10 is fed out of the device 1. In the second embodiment of the invention, shown in FIG. 2, the web feeding out of the device 1 is a multilayer web 19 comprising the meltblown composite web 8 interposed between the first continuous web 14 of non-woven fabric, specifically spunbond, and the second web 16 of non-woven fabric, specifically spunbond.

The meltblown composite web 8 or the multilayer web 19 comprising the meltblown composite web 8 will subsequently be cut into respective pieces to define the absorbent pad of an absorbent article.

The meltblown composite web 8 or the multilayer web 19 will subsequently define a roll of meltblown composite web 8 or a roll of multilayer web 19.

What is claimed is:

1. A device for forming a composite web of an absorbent pad for absorbent articles, such as diapers, for example, comprising an extruding means for extruding a thermoplastic material and which is provided with one or more channels, each having a respective delivery mouth for delivering filaments of thermoplastic material;
a delivery means for delivering superabsorbent polymer material has at least one outlet mouth for feeding the superabsorbent polymer material into the flow of filaments of thermoplastic material coming out of the extruding means;
a suction forming surface is configured to receive the filaments of thermoplastic material coming out of the extruding means and comprising the superabsorbent polymer material, to make a composite, meltblown web comprising meltblown thermoplastic fibers and superabsorbent polymer material;
characterized in that
the outlet mouth of the delivery means is configured to be movable at least from a first distance to a second distance, or vice versa, relative to the delivery mouth of the extruding means in such a way as to vary the concentration of the superabsorbent polymer material in the composite web.

2. The device according to independent claim 1, wherein the suction forming surface is the peripheral surface of a suction roller that rotates about its axis.

3. The device according to claim 1, wherein it comprises a first feed unit for feeding a first continuous web of non-woven fabric, preferably spunbond; the first feed unit being disposed upstream of the forming surface, in the feed direction of the composite web, so that the meltblown composite web is made on the first continuous web.

4. The device according to claim 1, wherein it comprises a second feed unit for feeding a second continuous web of non-woven fabric, preferably spunbond; the second feed unit being disposed downstream of the forming surface, in the feed direction of the composite web, so that the second continuous web is disposed on the composite web.

5. The device according to claim 1, wherein it comprises a feeding means for feeding a moistening substance and disposed downstream of the second feed unit, in the feed direction of the second continuous web.

6. The device according to claim 1, wherein it comprises a feeding means for feeding a moistening substance and configured to feed the extruding means.

7. The device according to claim 1, wherein it comprises a pressurized fluid source connected to the delivery means so that the superabsorbent polymer material is fed under pressure into the flow of filaments of thermoplastic material coming out of the extruding means.

8. A method for making a composite web of an absorbent pad for absorbent sanitary articles, such as diapers, for example, using the device according to claim 1, wherein it comprises a step of varying the position of the outlet mouth of the delivery means relative to the delivery mouth of the extruding means so as to vary the concentration of the superabsorbent polymer material in the composite web.

9. The method according to claim 8, wherein the composite web is made on top of the first continuous web of non-woven fabric.

10. The method according to claim 8, wherein the second continuous web of non-woven fabric is deposited on top of the composite web.

11. An absorbent sanitary article comprising at least one piece of composite web made using the device according to claim 1.

12. An absorbent sanitary article comprising at least one piece of a multilayer web comprising at least the composite web made using the device according to claim 1, interposed between a first continuous web of non-woven fabric, specifically spunbond, and a second continuous web of non-woven fabric, specifically spunbond.

13. A roll comprising a composite web made using the device according to claim 1.

14. A roll comprising multilayer web comprising at least the composite web made using the device according to claim 1, interposed between a first continuous web of non-woven fabric, specifically spunbond, and a second web of non-woven fabric, specifically spunbond.
